(19) United States
(12) Patent Application Publication
(10) Pub. No.: US 2025/0073393 A1
(43) Pub. Date: Mar. 6, 2025

(54) REFILL KIT FOR IMPLANTABLE PUMP

(71) Applicant: AMNEAL PHARMACEUTICALS, LLC, Bridgewater, NJ (US)

(72) Inventors: Walter Lee Nash, Brookhaven, GA (US); Scott William Spencer, Fenton, MO (US)

(73) Assignee: AMNEAL PHARMACEUTICALS, LLC, Bridgewater, NJ (US)

(21) Appl. No.: 18/240,029

(22) Filed: Aug. 30, 2023

(51) Int. Cl. A61M 5/24

(52) U.S. Cl. CPC A61M 5/24; A61M 2209/045

(57) ABSTRACT

The invention relates to a kit for refilling an implantable pump and, more particularly, to packaging and contents of a kit to improve the workflow of a user while adding medication to an implantable pump that has been surgically implanted in a patient. kit for refilling an implanted pump. The kit comprises a setup tray containing at least one needle, a fenestrated drape, a first syringe, a needle, a filter, and clampable tubing; and a medication tray containing a second syringe, at least one ampule of medication, an ampule opener, and aspiration tubing. The medication tray is molded to have a recess that securely holds the at least one ampule in both a horizontal position and in a vertical position.

### TECHNICAL FIELD

The invention relates to a kit for refilling an implantable pump and, more particularly, to packaging and contents of a kit to improve the workflow of a user while adding medication to an implantable pump that has been surgically implanted in a patient.

### BACKGROUND

Implantable pumps are medical devices designed to deliver drugs directly into a patient's bloodstream or a specific target site in the body. These pumps are surgically implanted under the skin and are typically controlled remotely by a healthcare professional. Implantable pumps can be programmed to deliver drugs continuously or in a specific schedule, depending on the provider's treatment plan. The drugs are typically stored in a reservoir inside the pump and are delivered through a catheter or tube that is inserted into the body. Implantable pumps are commonly used for pain management, chemotherapy, and the treatment of chronic conditions such as Parkinson's disease and spasticity. They are typically implanted under the skin in the patient's abdomen.

When the pump is activated, the drug is pushed out of the reservoir and into the catheter, which delivers it to the targeted site. The rate and amount of drug delivered can be adjusted by the healthcare provider allowing for precise dosing and control. The adjustment typically is done through a wireless programming device that communicates with a memory or processor in the pump when the programming device antenna is placed on the skin of the patient proximate to the pump.

Implantable pumps that are used to deliver drugs need to be refilled periodically to ensure that the medication supply does not run out. The frequency of refilling depends on the type of pump, reservoir size, and the dosage requirements/schedule of the patient. To refill an implantable pump, a healthcare provider typically uses a needle to access the refill port (which typically includes a self-sealing septum) of the pump through the patient's skin. The refill port is connected to the drug reservoir inside the pump. The healthcare provider will withdraw any remaining medication from the reservoir, using the same needle, and then refill the reservoir with the appropriate medication. Once the reservoir is refilled, the healthcare professional will remove the needle and close the refill port.

The refill process is usually performed in a healthcare setting, such as a hospital or clinic, by a trained healthcare professional. Patients who have implantable pumps are often given instructions on how to monitor the medication levels in their pump and when to schedule a refill appointment. Because the refill process requires penetrating the patient skin to access the refill port septum, it is critical that the process be done as sterile as possible to minimize risk of infection or contaminating the pump reservoir. Refilling is typically done using disposable supplies that must be kept sterile. There is a need to simplify the refilling process while keeping the tools and administration area of the patient sterile. This can reduce negative outcomes while reducing the skill level of the user of the refill user.

One common case of an implantable pump and the need to refill the pump occurs with baclofen. Baclofen is a medication that is commonly used to treat spasticity, a condition characterized by involuntary muscle contractions and stiffness. When oral medications are not effective or cause intolerable side effects, baclofen can be delivered directly into the spinal fluid through an implantable intrathecal pump.

An intrathecal pump is a small device that is surgically implanted under the skin (typically in the abdomen) and connected to a catheter that is threaded into the spinal fluid in the intrathecal space. The pump is programmed to deliver a precise amount of baclofen directly into the spinal fluid at a controlled rate. By delivering the medication directly into the spinal fluid, the intrathecal pump can achieve a more targeted effect with a lower dose of medication than would be required with oral administration. This can help reduce side effects and improve overall effectiveness. The pump can be programmed to deliver a continuous infusion of baclofen or to administer the medication at specific times of the day or in response to certain triggers, such as muscle spasms. The dosage and delivery schedule can be adjusted by a healthcare professional to achieve optimal therapeutic effect. The pump includes a battery that is typically expected to last several years and a reservoir that is typically 20-40 ml. Typical dosing regimens will deplete this volume in a few weeks or a few months. Therefore, the pump will commonly need to be refilled several times during its lifetime.

### SUMMARY

In one embodiment, the present disclosure is directed to a kit for refilling an implanted pump comprising a carton, a first sealed tray contained in the carton, and a second sealed tray contained in the carton. The first sealed tray containing at least one needle, a fenestrated drape, a first syringe, a needle, a filter, and clampable tubing; and the second sealed tray containing a second syringe, at least one ampule of medication, an ampule opener, and aspiration tubing. The second sealed tray is molded to have a recess that securely holds the at least one ampule in both a horizontal position and in a vertical position.

In one embodiment, the implanted pump is an intrathecal baclofen pump.

In one embodiment, the at least one ampule of medication is a 5 ml ampule comprising 2000 mcg/ml baclofen. In one embodiment, the kit comprises two 5 ml ampules comprising 2000 mg/ml baclofen. In one embodiment, the kit comprises one 20 ml ampule comprising 2000 mcg/ml baclofen.

In one embodiment, the at least one needle is a 22 gauge needle. In one embodiment, the first sealed tray comprises two needles. In one embodiment, the two needles are 22 gauge needles with 1.5 inch and 2 inch length.

In one embodiment, the first and the second sealed trays are sealed with a polyethylene based film. In one embodiment, the polyethylene based film is compatible with commonly used sterilization methods selected from the group consisting of sterilization with ethylene oxide, gamma rays, and electron-beam. In one embodiment, the polyethylene based film is compatible to sterilization with ethylene dioxide.

In one embodiment, the first tray and the second tray are sterilized after sealing such that the ampule, the first syringe, and the second syringe remain sterile during refilling of the implanted pump.

In another embodiment, the disclosure is directed to kit for refilling an implanted pump comprising a carton, a first sealed tray contained in the carton, and a second sealed tray contained in the carton. The first sealed tray containing at least one needle, a fenestrated drape, a first syringe, a needle, a filter, and clampable tubing; and the second sealed tray containing a second syringe, at least one ampule of medication, an ampule opener, and aspiration tubing. Each of the first and the second sealed tray is sterilized after sealing.

In one embodiment, the implanted pump is an intrathecal baclofen pump.

In one embodiment, the first sealed tray comprises 2 needles. In one embodiment, the two needles are 22 gauge needles with 1.5 inch and 2 inch length.

In one embodiment, the first and the second sealed trays are sealed with a polyethylene based film. In one embodiment, the polyethylene based film is compatible to sterilization with ethylene dioxide.

In one embodiment, the disclosure provides a kit to facilitate easy, efficient, and sterile refilling of baclofen in an implantable intrathecal pump. The kit comprises a sealed setup tray comprising items for removing any residual baclofen from the pump, and a sealed medication tray comprising items for refilling the pump with fresh baclofen. The sealed setup tray comprises layers in the following order:
a) a first setup tray layer facing the user and comprising a topical antiseptic solution wipes and applicators,
b) a second setup tray layer positioned below the first setup tray layer and comprising sterile disposable gloves,
c) a third setup tray layer positioned below the second setup tra layer and comprising a fenestrated drape, and
d) a fourth setup tray layer positioned below the third setup tray layer and comprising a syringe, a needle, clampable tubing, and a sharps plug.

The sealed medication tray comprises layers in the following order:
a) a first medication tray layer facing the user and comprising a syringe, prescribing information, and information for use,
b) a second medication tray layer positioned below the first medication tray layer and comprising at least gauze pad or a gauze roll,
c) a third medication tray layer positioned below the second medication tray layer and comprising at least one polybag, and
d) a fourth medication tray layer beneath the third medication tray layer comprising at least one gauze pad or a roll and a label,
e) a fifth medication tray layer positioned below the fourth layer and comprising a medication ampule, aspiration tube, ampule opener, and a syringe cap.
The setup tray and the medication trays are sterilized after sealing to keep the outer surface of the ampule and the syringes sterile during use. The order of layers in the setup tray and the medication tray is based on requirement of items during each step of refilling the pump, such that the items for an immediate step should be immediately available to a user.

### FIGURES

The accompanying drawings, which are incorporated in and form a part of the specification, illustrate the embodiments of the invention and together with the written description serve to explain the principles, characteristics, and features of the invention. In the drawings:

FIG. 1 shows an exemplary kit with a divider 14 separating a medication tray 50 and a setup tray 20.

FIG. 2 is a perspective view of an exemplary sealed medication tray (also referred to as drug preparation kit).

FIG. 3 is a perspective view of an exemplary sealed setup tray (also referred to as a refill kit).

FIG. 4 shows a first layer (top layer facing the user) of the setup tray comprising topical antiseptic solutions and applicators to disinfect skin, such as alcohol wipes 23 and povidone-iodine swabs 24.

FIG. 5 shows the second layer of the setup tray. The second layer is positioned under the first layer of the setup tray. The second layer comprises latex-free/nitrile disposable gloves 26 to use during the drug emptying and refill procedure.

FIG. 6 shows the third layer of the setup tray. The third layer is positioned under the second layer of the setup tray. The third layer comprises one or more sterile drapes, including a fenestrated drape 27.

FIGS. 7 and 8 show the fourth layer of the setup tray. The fourth layer is positioned under the third layer of FIG. 6. The fourth layer comprises additional supplies needed to access the pump reservoir and to remove any old medication in preparation for the refill. These contents are generally held in place by molded recesses.

FIG. 9 shows the recesses of the setup tray body in further detail.

FIG. 10 shows the first layer (top layer facing the user) of the medication tray (drug prep kit) comprising a 20 mL (or larger, if needed) syringe, drug product information (PI), and drug instructions for use (IFU). The syringe will be used to aspirate fresh medication from ampules provided.

FIG. 11 shows the second layer of the medication tray. The second layer is positioned under the first layer of the medication tray. This layer comprises 2-4 rolls or stacks of gauze.

FIG. 12 shows the third layer of the medication tray. The third layer is positioned under the second layer of the medication tray. The third layer comprises a zip top plastic bag, and a syringe label to include patient information.

FIG. 13 shows the fourth layer of the medication tray. The fourth layer is positioned under the third layer of the medication tray. This layer comprises medication ampules 62, ampule opener 64, filter straw/aspiration tubing 66, and syringe cap 68. These contents are generally held in place by molded recesses.

FIG. 14 shows the details of the various recesses that hold the contents of the fourth layer in FIG. 15. These include one or more ampule recesses to hold the ampules in a horizontal position until removed, a recessed vertical holder integrated into each of these recesses.

FIG. 15 shows vertical holders 72 holding ampules 62 upright.

FIG. 16 shows detailed view of vertical holders 72 and corrugations 76 molded into the walls of recess 70 and coincident with vertical holder 72.

### DESCRIPTION

This disclosure is not limited to the particular systems, devices and methods described, as these may vary. The terminology used in the description is for the purpose of describing the particular versions or embodiments only and is not intended to limit the scope.

Embodiments disclosed herein create a kit of medical supplies and medication that facilitates easy, efficient, and sterile refilling of an implantable pump, such as an intrathecal baclofen pump.

When refilling an implantable or intrathecal pump, a healthcare provider will generally follow a specific set of steps to ensure that the process is safe and effective. These steps include the following.

Procedure preparation: The healthcare provider will first review the patient's medical history and the medication requirements, as well as check the pump's settings and refill history. This can include interrogating the pump using a handheld near-field wireless programming device provided by the pump manufacturer. They will then prepare the refill equipment, which typically includes at least a sterile syringe, needle, sharps plug, extension set or catheter, filter straw/aspiration tubing, filter, and one or more medication vials/ampules. This equipment will be discussed in more detail later.

Patient positioning: The patient should be positioned appropriately to access the refill port, which is located under the skin. Depending on the location of the pump, the patient may need to lie on their stomach or back. A typical implantation places the pump in the patient's abdomen and requires the patient to sit or lie on their back during the refill process.

Preparing medication filled syringe: The healthcare provider will remove the setup tray 20 and the medication tray 50 from the refill kit. The provider will then open the setup tray; remove the sterile gloves; wear the sterile gloves; remove the sterile towel drape and place it on the work surface. The provider will open the medication tray; read the prescribing information (PI) and information for use (IFU); tilt the ampule 62 from the horizontal position to a secured vertical position; place the ampule opener 64 on the neck of ampule; push the opener to the scored line of the ampule; snap the ampule top; discard the ampule top in sharps plug 35; assemble the syringe and filter straw/aspiration tubing 66 together and withdraw the drug from the ampule into the syringe through the aspiration tubing; remove any air from the syringe; close the syringe with a stopper 68; label the syringe with patient name, drug name, drug concentration, and date. The labeled syringe is finally placed in a plastic bag.

Anesthesia: The healthcare provider can optionally apply a local anesthetic or analgesic to the skin to minimize any discomfort during the refill process.

Locating the pump: Typically, the provider can locate the pump by palpation of the patient skin to locate the rigid pump housing beneath the softer patient flesh. A typical implantable pump design has a round, flat face that faces outward, allowing palpation to locate the extends of the face. The refill port septum is typically centered in this round face to allow the port to easily be located by finding the extents of the outward-facing housing face.

Preparing patient skin: The healthcare provider will first clean the skin around the refill port using an antiseptic solution, such as alcohol. The area will be cleaned thoroughly to remove any dirt, oil, or bacteria that could potentially contaminate the refill port and increase the risk of infection. This step can also include applying a fenestrated drape that covers the adjacent parts of the patient body to create a sterile environment around the target skin.

Accessing the refill port: The healthcare provider will use a sterile needle to access the refill port, which is typically located on the top or side of the pump. In some embodiments, locating the port can be aided by applying an adhesive template 30 that is aligned to the pump housing to indicate a near-exact approximation of the center of the refill port septum. The provider will then attach a syringe to the needle, typically via extension set or clampable tubing 34 or catheter.

Withdrawing remaining medication: The healthcare provider will withdraw any remaining medication from the pump's reservoir, which helps prevent overfilling. This is done by pulling out the plunger of an empty syringe, which will aspirate any remaining fluid medication from the reservoir of the pump, allowing refilling with only a de minimis volume of old medication in the reservoir.

Refilling the reservoir: The healthcare provider will then discard the syringe with remaining fluid medication from the reservoir pump and couple the medication filled syringe (mentioned above) containing the new medication to the extension set or catheter and needle that is already inserted into the pump port. The provider will then slowly inject the new medication into the reservoir through the syringe (taking care not to overfill the pump).

Removing the needle: Once the refill is complete, the healthcare provider will remove the needle and dispose it off in sharps plug 35.

Closing the refill port: The healthcare provider will then clean the refill port area and apply a sterile dressing or adhesive bandage (such as a Band-Aid) to protect the site and prevent infection.

Reprogramming the pump: Using the handheld near-field wireless programming device, the provider can update the pump's memory to reflect that the reservoir has been refilled to a specified volume and indicate information about the medication, such as type, brand, lot number, date, etc.

Monitoring: The healthcare provider will monitor the patient closely for any adverse reactions or complications, such as bleeding, infection, or leakage of the medication.

The specific steps involved in the refill process may vary depending on the type of pump and medication being used. It is generally important, at least in the prior art, for healthcare providers to receive specialized training in pump refill procedures and to follow established protocols to ensure safe and effective use of the device.

By simplifying the refilling process by providing the refill equipment in logical packaging and organized manner, some embodiments can allow an unskilled user (or one without specific training) to perform the refill procedure. In some embodiments, this can include caregivers who are not trained medical professionals. Thus, the term “user” refers to the person who is administering the refill procedure. This may be a healthcare provider, such as a doctor, nurse, technician, etc. It can also refer to a caregiver in the patient's home or the patient themselves if they feel comfortable undertaking the refill procedures.

In some embodiments, a kit contains two sterilized, sealed trays: a refill setup tray (also known as refill kit) and a medication tray (also known as drug preparation kit). These trays can be placed in the same cardboard box, in a stacked configuration or in adjacent separated compartments. Each tray can include a tray body and a sealing film/lid. For example, a molded or vacuum-formed plastic body can form the tray body, while a sealing film can be sealed to that body. In some embodiments, a tray includes a vacuum-formed styrene film to form the body and a sealing film is applied in a sealed manner to form a lid. In some embodiments, the sealing film comprises a breathable TYVEK® material. In some embodiments, the sealing film with breathable TYVEK® material is a TYVEK® film. In some embodiments, the Tyvek film is ideal for providing a breathable and microbial barrier seal. In some embodiments, the TYVEK® film exhibits excellent tear strength and puncture resistance. In some embodiments, the TYVEK® film is compatible with commonly used sterilization methods comprising sterilization with ethylene oxide, gamma rays, electron-beam, etc. In some embodiments, the TYVEK® film is used as a lidding material for rigid trays. In some embodiments, the kit also includes a sterile work surface drape that allows a sterile work surface to be created on any flat surface.

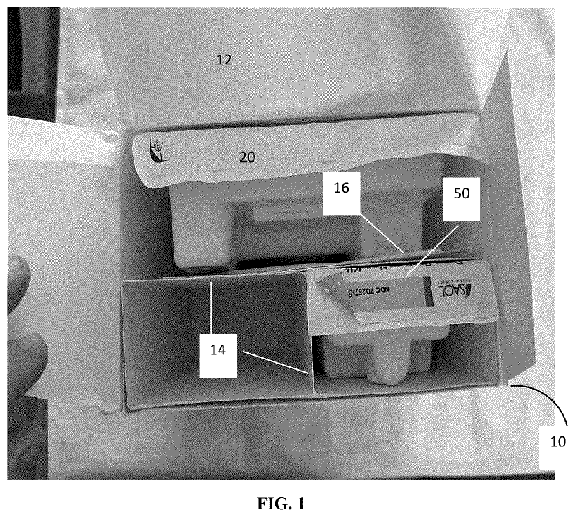

FIG. 1 shows exemplary kit 10 for an implanted pump. It includes a carton 12 (e.g., cardboard), which may include one or more dividers 14 to separate two trays: a setup tray 20 and a medication tray 50. In some embodiments, the carton may include a work surface drape 16. In some embodiments, more than one medication tray can be included depending on the amount of medication needed to refill the pump. Each tray is sterilized in a sealed manner. Exemplary trays include a rigid tray body and a sealing film cover, such as a flash-spun HDPE file (e.g., TYVEK®) that is bonded to the tray. In some embodiments, the rigid tray body can be placed in a sealed envelope made of a suitable material, such as TYVEK®, rather than having a film sealed directly to the tray. Some embodiments include a pre-packaged work surface drape 16 in the carton, separate from the trays; in some embodiments, the drape is provided in setup tray 20. In some embodiments, the setup tray 20 includes a fenestrated drape 27 that covers the adjacent parts of the target skin on the patient body to create a sterile environment around the target skin. Carton 12 can also include instructions on a label or as a folded booklet in the carton.

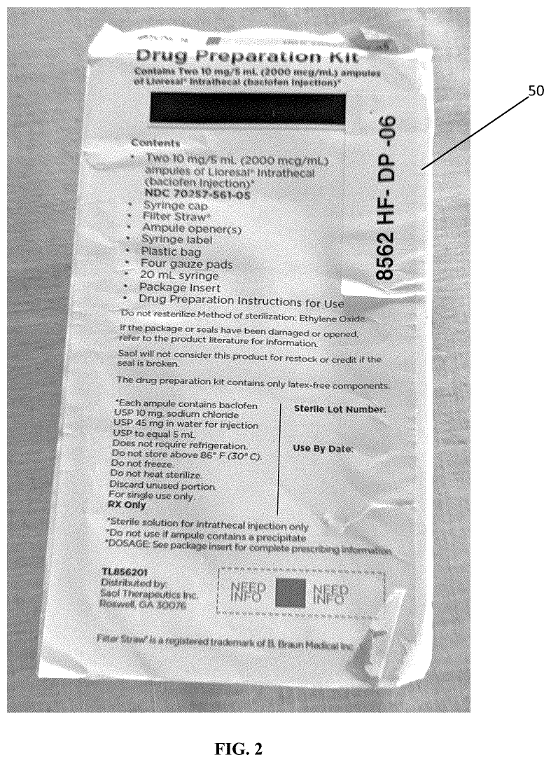

FIG. 2 is a perspective view of an exemplary sealed medication tray 20 (which may be referred to as a drug preparation kit).

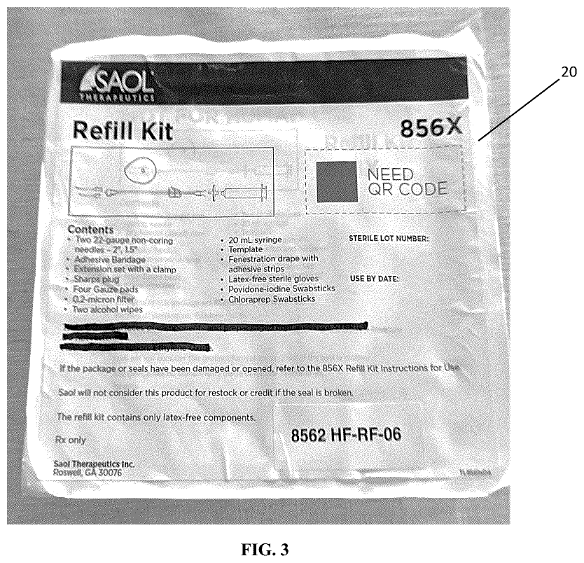

FIG. 3 is a perspective view of the exemplary sealed setup tray 50 (which may sometimes be referred to as a refill kit).

These two trays include contents that have been packed in logical layers according to their order of use. The bottommost layer of supplies generally contains the last items to be used and can be held in place by recesses formed in the tray body to keep those contents in place. In some embodiments, these trays are formed from vacuum-molded styrene, which include molded recesses to secure contents of the bottommost layer. Exemplary methods for forming the tray bodies or inserts within the tray body to secure the contents include injection molding, vacuum forming, die cutting, and 3D printing. The recesses for securing contents can be loose-fitting or tight-fitting for the contents, as appropriate (e.g., the recesses can have tight fitting features to secure glass ampules of medication while being loose for less sensitive contents, such as tubing). Recesses can include tabs or finger-grip recesses that allow fingers to reach under the contents to make content removal easier.

Once contents are packed into the tray at manufacture, the tray is sterilized, and sealed with a film, such as solid polymer film (such as polyethylene, polyester, or polypropylene) or a solid, woven, or spun high density polyethylene (HDPE) film. In some embodiments, the sealed film comprises a TYVEK® film comprising a 100% synthetic material made from high-density spun bound polyethylene fibers. The TYVEK® film is lightweight, durable and breathable, yet resistant to water, abrasion, bacterial penetration and aging. Bonding between the film and tray body can be accomplished through a variety of methods, including heat sealing, adhesive bonding, and ultrasonic welding. Exemplary sterilization methods include ethylene oxide (EO) sterilization, gamma irradiation, electron beam sterilization, and steam sterilization. The exact materials and sterilization methods chosen can depend on the exact nature of the refill kit.

These layers will be explained from the user perspective. Generally, they are layered so that supplies for each step in the pump-refill process will be immediately available at the top-most remaining layer such that supplies can remain sterile, in the tray until needed. This eliminates the need to lay out supplies on another surface to organize them. In some embodiments, the user will remove several items from the tray together and can use the sterile drape work surface to facilitate organization and use for a given step.

Other design considerations include the following. The trays and carton packaging should include labels that a user can understand. Users are typically trained in a hospital environment or health care provider (HCP) office, and labels should be consistent with industry practices. For example, the refill kit packaging should clearly identify important information, such as the type of medication, concentration, volume, and expiration date. All packaging should include seals that can be easily inspected to verify that the integrity of packaging has not been compromised. Labels should clearly identify contents, allowing users to easily ascertain which components that supplies are in each tray. The cover films and the labels thereon should be labeled to clearly identify the trays as setup tray and medication tray and how they will be used.

To begin the refill procedure, the user should remove trays and open each tray by pulling off/back the sealed cover. The user removes any instructions in the carton or sealed with the contents of the medication tray 50 and the setup tray 20 and familiarizes himself with the procedure. The carton can also include a work surface drape that can be removed and placed on a work surface. This drape can be placed on a table to provide a clean or sterile work area.

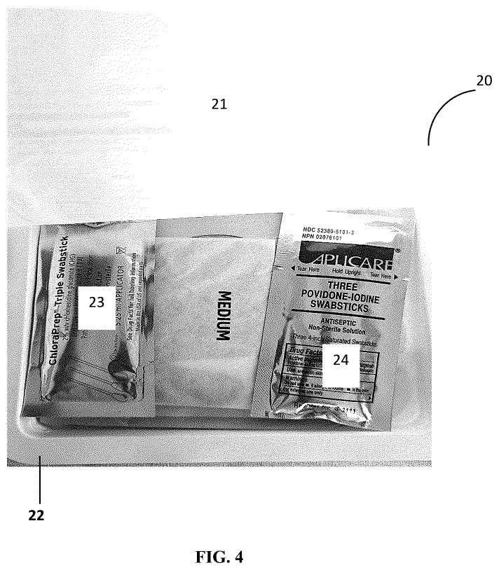

FIGS. 4-9 show the various layers of disposable supplies in the setup tray of the kit that a user will find upon unsealing the exemplary sealed setup tray 20. FIG. 4 shows the first layer (facing the user) of the setup tray 20. The contents of the setup tray facilitate creating access to the pump reservoir and aspirating any remaining fluid before refilling with fresh medication. A user gains access to the contents of setup tray 20 by peeling cover film 21 from tray body 22. This topmost layer/first layer facing the user includes topical antiseptic solutions and applicators to disinfect skin, such as alcohol wipes 23 and povidone-iodine swabs 24. These can be used to cleanse the patient skin around the pump to limit the risk of infection. These items can be placed on the draped work surface before use.

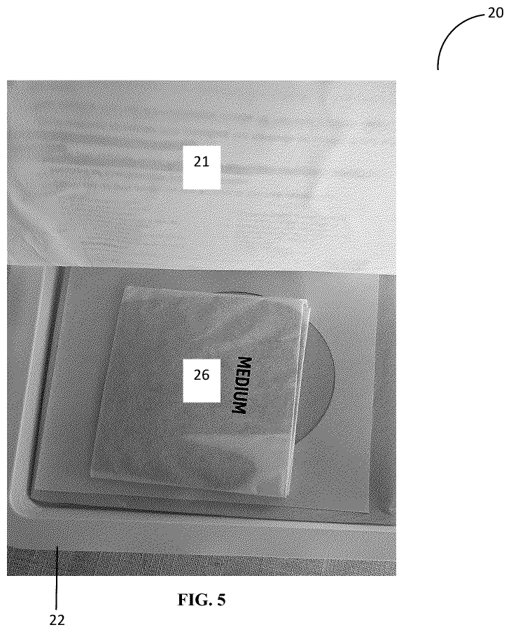

FIG. 5 shows the second layer positioned below the first layer of the setup tray. It includes latex-free/nitrile disposable gloves 26 to use during the procedure. These can also be placed on the draped work surface or used immediately with antiseptics. The user should palpate the area of skin around the pump to determine the location of the pump beneath the skin. The user can then align a near-field programming device (not included in the refill kit) to interrogate the pump. Then, the user should don the gloves and use disinfectants 23 and 24, in accordance with manufacturer directions. This will disinfect the patient skin and ensure the user has sterile hands. The first and second layers can be considered a single logical layer, as the contents will be used simultaneously.

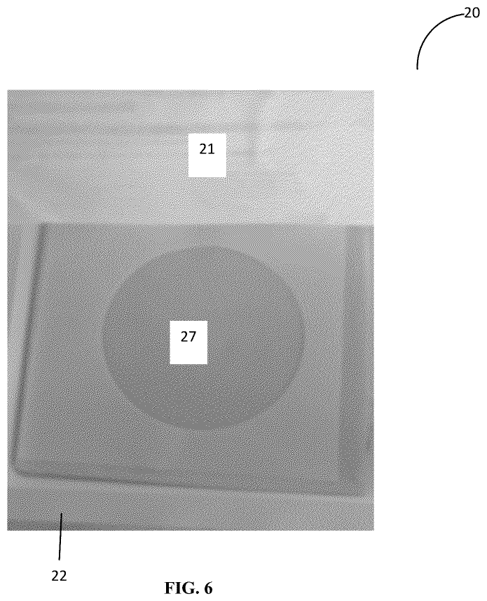

FIG. 6 shows the third layer positioned below the second layer of the setup tray. The third layer includes one or more sterile drapes, including a fenestrated drape 27. This can be placed over the patient's body adjacent to the pump such that the hole of the drape exposes the disinfected skin around the pump. Fenestrated drape 27 can include adhesive tape around the hole that can secure the drape to the patient skin so that it remains in place throughout the procedure, minimizing injection-site contamination.

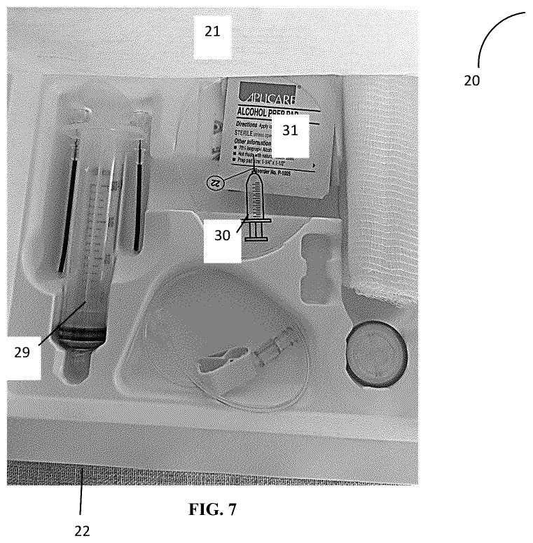

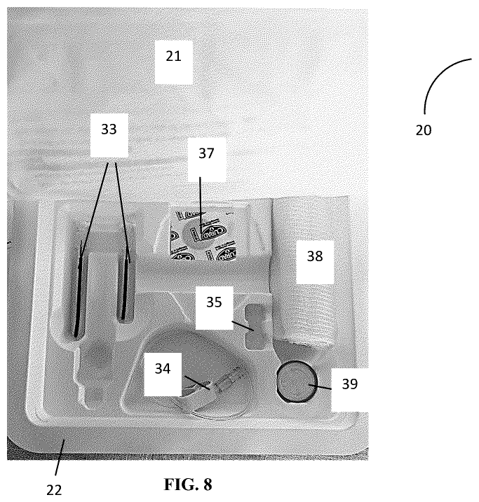

FIGS. 7 and 8 show the fourth layer positioned below the third layer of the setup tray. The fourth layer includes the remaining supplies needed to access the pump reservoir and to remove any old medication in preparation for the refill. These contents are generally held in place by molded recesses. As can be seen, most of the contents in this layer can generally be accessed in parallel, but some contents can also be stacked within this layer to better contain the supplies. For example, a single recess contains a template 30 above one or two alcohol wipe(s) 31 (which can be used to remove any adhesive residue from the patient skin once template 30 and fenestrated drape 27 are removed at the end of the procedure), a small adhesive bandage 37 (which can be applied after the procedure to stop any bleeding once a needle is removed). This fourth layer includes a syringe 29 (which will be used to aspirate any old medication), one or more needles 33 (in some embodiments, such as shown, multiple lengths sizes of needles can be provided to accommodate pump implantations that may be at different subcutaneous depths in different patients), clampable tubing 34 (which includes a clamp or pinch valve to allow syringe 29 and the medication syringe, explained later, to be coupled to and uncoupled from the needle without disturbing the needle), gauze 38 (which can be used for general cleanup during the procedure) and filter 39 (which can be added to the syringe end of clampable tubing 34 when medication is added later). The fourth layer can also include sharps plug 35 (which can be slid onto the tip of the used needle at the end of the procedure),

The user should remove the tray contents and apply the template to the patient skin. This template is a clear plastic film with printed markings, and a hole in the center. The outer edge is generally the same shape and size as the subdermal pump. By palpation, the user can locate the extents of the subdermal pump and align the template with the skin in front of the pump. A typical pump is round with a concentrically located catheter access port. The hole in the template aligns with the location of the access port beneath the skin.

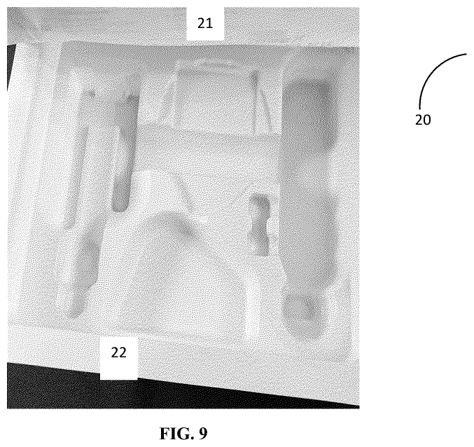

The user then selects the appropriate needle size for the patient and pump, couples needle 33 to one end of clamp tubing 34 and syringe 29 to the other end of the clamp tubing 34. The coupling can be any suitable form, such as a slip fit or a Luer lock. The user then inserts needle 33 into the patient skin at the location of the hole of template 30, which allows the needle to pierce the skin and penetrate the diaphragm of the subdermally-located refill port of the pump. The user then withdraws the plunger of syringe 29 to aspirate any remaining medication from the pump reservoir, allowing the reservoir to be substantially emptied before refilling with fresh medication. The clamp of tubing 34 is then locked to prevent any air from filling the tubing. Syringe 29 is twisted (in the case of a Luer lock) to unlock from tubing 34 and discarded. Filter 39 is then attached to the medication syringe 55 before being attached to the clampable tubing 34 prior to administration of new drug. FIG. 9 shows the recesses of tray body 22 in further detail.

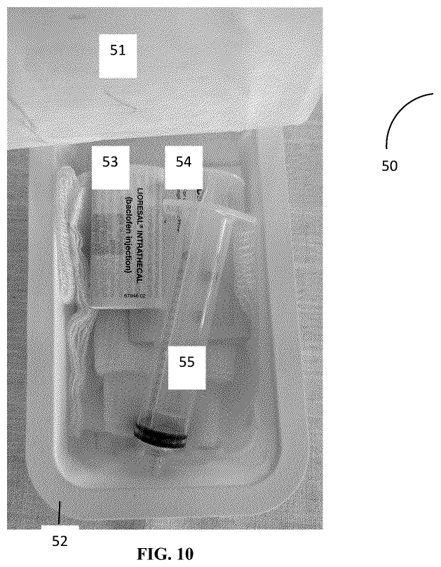

FIGS. 10-13 show the various layers of disposable supplies in the medication tray (drug preparation kit) 50 that a user will find upon unsealing the exemplary sealed medication tray.

FIG. 10 shows the first layer (top layer facing the user) of medication tray 50. This includes the first items needed in the refill process upon removing the sealing film lid 51 from tray body 52 (which opens the sterile contents to the air). A 20 mL (or larger, if needed) syringe 55 is provided, along with necessary information and instructions, such as drug product information (PI) 53 and drug instructions for use (IFU) 54. The user should read this information before proceeding. Syringe 55 will be used to aspirate fresh medication from ampules provided.

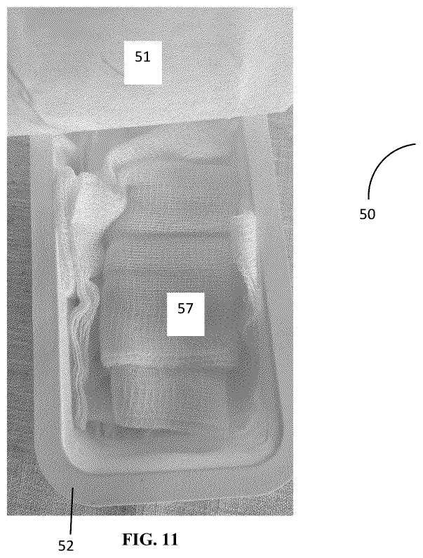

FIG. 11 shows the second layer of the medication tray. The second layer is positioned below the first layer of the medication tray. This layer includes 2-4 rolls or stacks of gauze 57. These can be used for cleanup during the procedure. In some embodiments, pre-packaged gauze pads can be provided instead. The contents of the layers shown in FIGS. 10 and 11 can be removed and placed on the draped work surface.

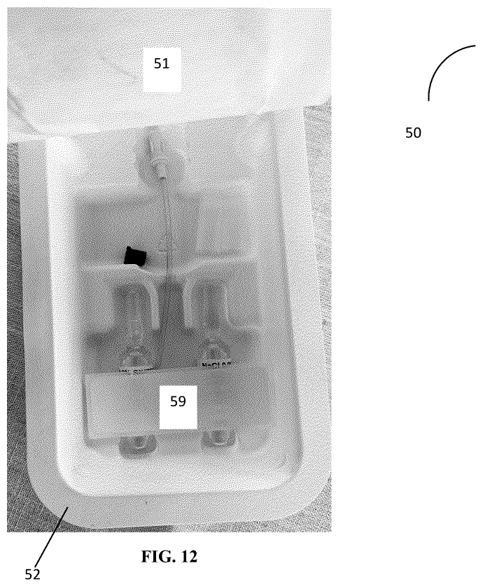

FIG. 12 shows the third layer positioned below the second layer of the medication tray. The third layer includes a zip top plastic bag (not shown in the figure) and a syringe label 59. The plastic bag can be used to gather used components and aid in cleanup or store the prefilled syringe with fresh medication for administration. Label 59 can include patient information, medication name, and strength. In some embodiments, label 59 can be applied to plastic bag containing syringe 55. In some embodiments, label 59 can be applied to the medication syringe 55 with fresh medication.

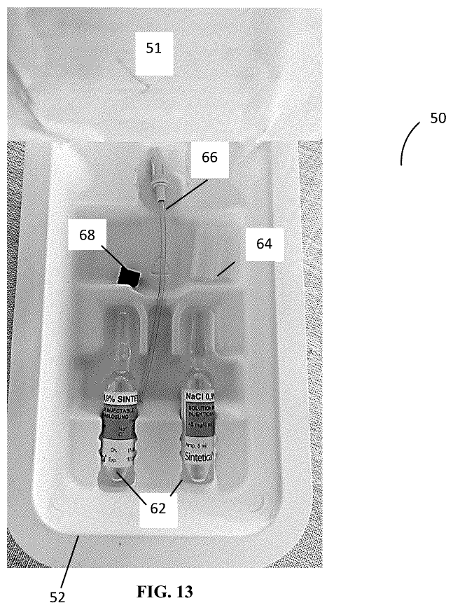

FIG. 13 shows the fourth layer positioned below the third layer of the medication tray. This layer includes the bottommost contents of medication tray 50. These contents are generally held in place by molded recesses. This layer includes medication ampules 62, ampule opener 64, filter straw/aspiration tubing 66, and syringe cap 68. The size and contents of ampules 62 will be based on the patient prescription. In this figure, two 5 mL ampules of baclofen at shown. These are glass ampules and can be opened using ampule opener 64. Ampule opener 64 is a rigid plastic cylinder with an internal concentric flange located near one end. This flange can include radial gaps allowing it to slip over the bulbous head of a glass ampule to be placed concentric to the neck of the ampule. Once positioned, the opener axis can be torqued relative to the axis of the ampule, allowing the glass to be cleanly and safely snapped at the neck. This gives the user access to the contents of the ampule without requiring a separate cap.

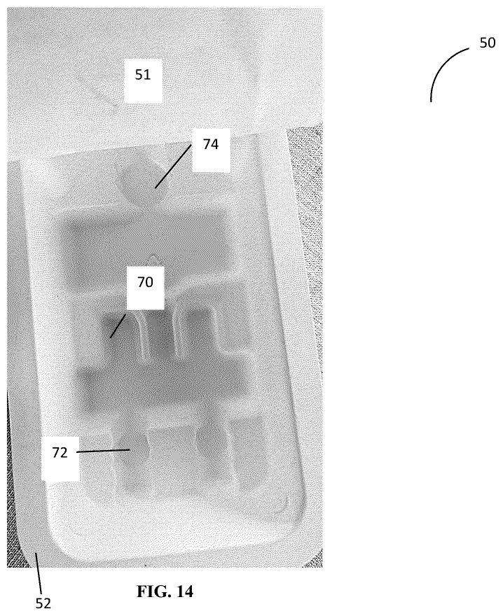

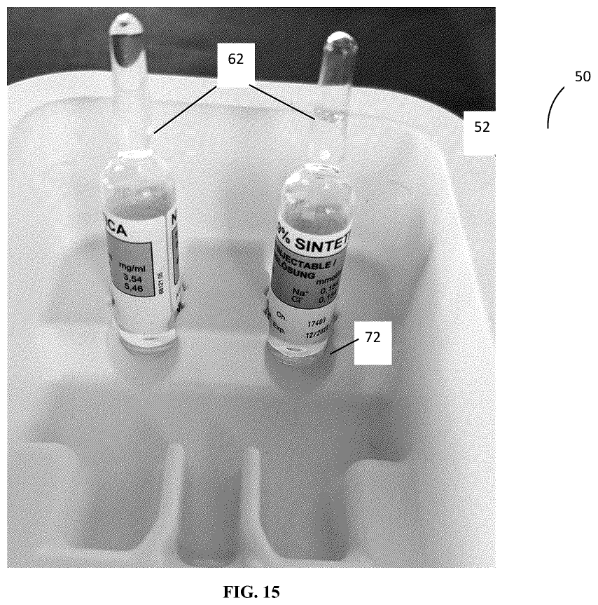

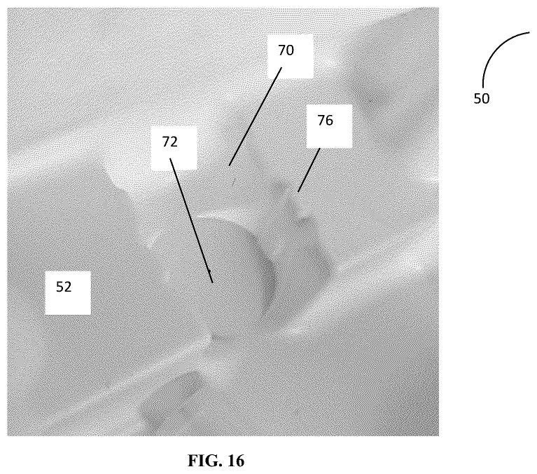

FIG. 14 shows the details of the various recesses that hold the contents of FIG. 13. These include one or more ampule recesses 70 that hold the ampules 62 in a horizontal position until removed. Integrated into each of these recesses, is a recessed vertical holder 72. In some embodiments, a single tray body 52 is molded so that it can hold different sized ampules, to allow for larger dosages to be contained. Tray body 52 can include a larger recessed vertical holder 74. Vertical holders 72 and 74 have similar construction and function. Vertical holders 72 are shown in more detail in FIG. 15, holding ampules 62 upright. Vertical holder 72 is shown in more detail in FIG. 16, which illustrates how corrugations 76 can be molded into the walls of recess 70 coincident with vertical holder 72. In this figure, tray body 52 is vacuum-formed styrene, which allows corrugations 76 to be rigid enough to securely hold in the ampule in either orientation and also to be compliant enough to easily press the ampule into position.

To use the contents of medication tray 50, a user will peel back lid 51 from body 52. Syringe 55 can be removed and placed on the draped work surface while the user reads instructions and disclosures in PI 53 and IFU 54. Gauze 57, bag 58, and label 59 can be set aside for subsequent cleanup. The user then removes ampules 62 and opens them using opener 64. These ampules can then be placed upright in the vertical holders 72 by pressing the base of each ampule down through corrugations 76 until the bottom of the ampule rests on the flat base of tray body 52.

The user then removes aspiration tubing 66 from the tray and couples it to the tip of syringe 55, such as by screwing a Luer lock coupling. The user then inserts the free end of the tubing into each ampule and aspirates the required medication amount from the ampule(s). The user then decouples aspiration tubing 66 and disposes it. The user couples the filled medication syringe 55 to filter 39 and purges air. The user then couples the other end of filter 39 to the free end of clamped tubing 34 and unclamps it. Syringe 55 now has a fluid connection to the pump reservoir with an inline filter to avoid pushing glass or foreign bodies into the pump. The user presses the plunger of the syringe to inject the medication into the pump. The user then clamps the tubing, carefully removes needle 33 from the patient, applies adhesive bandage 37, caps needle 33 with sharps plug 35 before discarding. The supplied gauze can be used for any incidental cleanup. Template 30 and fenestrated drape 27 are removed and alcohol wipes can be used to clean the patient's skin.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure that are within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

A second action can be said to be “in response to” a first action independent of whether the second action results directly or indirectly from the first action. The second action can occur at a substantially later time than the first action and still be in response to the first action. Similarly, the second action can be said to be in response to the first action even if intervening actions take place between the first action and the second action, and even if one or more of the intervening actions directly cause the second action to be performed. For example, a second action can be in response to a first action if the first action sets a flag and a third action later initiates the second action whenever the flag is set.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 components refers to groups having 1, 2, or 3 components. Similarly, a group having 1-5 components refers to groups having 1, 2, 3, 4, or 5 components, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A kit for refilling an implanted pump comprising:
a carton;
a first sealed tray contained in the carton, the first tray containing at least one needle, a fenestrated drape, a first syringe, a needle, a filter, and clampable tubing; and
a second sealed tray contained in the carton, the second tray containing a second syringe, at least one ampule of medication, an ampule opener, and aspiration tubing,
wherein the second sealed tray is molded to have a recess that securely holds the at least one ampule in both a horizontal position and in a vertical position.

2. The kit of claim 1, wherein the implanted pump is an intrathecal baclofen pump.

3. The kit of claim 1, wherein the at least one ampule of medication is a 5 ml ampule comprising 2000 mcg/ml baclofen.

4. The kit of claim 1, wherein the kit comprises two 5 ml ampules comprising 2000 mg/ml baclofen.

5. The kit of claim 1, wherein the kit comprises one 20 ml ampule comprising 2000 mcg/ml baclofen.

6. The kit of claim 1, wherein the at least one needle is a 22 gauge needle.

7. The kit of claim 1, wherein the first sealed tray comprises two needles.

8. The kit of claim 7, wherein the two needles are 22 gauge needles with 1.5 inch and 2 inch length.

9. The kit of claim 1, wherein the first and the second sealed trays are sealed with a polyethylene based film.

10. The kit of claim 9, wherein the polyethylene based film is compatible with commonly used sterilization methods selected from the group consisting of sterilization with ethylene oxide, gamma rays, and electron-beam.

11. The kit of claim 9, wherein the polyethylene based film is compatible to sterilization with ethylene dioxide.

12. The kit of claim 1, wherein the first tray and the second tray are sterilized after sealing such that the ampule, the first syringe, and the second syringe remain sterile during refilling of the implanted pump.

13. A kit for refilling an implanted pump comprising:
a carton
a first sealed tray contained in the carton, the first tray containing at least one needle, a fenestrated drape, a first syringe, a filter, and clampable tubing; and
a second sealed tray contained in the carton, the second tray containing a second syringe, at least one ampule of medication, an ampule opener, and aspiration tubing,
wherein each of the first and the second sealed tray is sterilized after sealing.

14. The kit of claim 13, wherein the implanted pump is an intrathecal baclofen pump.

15. The kit of claim 13, wherein the first sealed tray comprises 2 needles.

16. The kit of claim 15, wherein the two needles are 22 gauge needles with 1.5 inch and 2 inch length.

17. The kit of claim 13, wherein the first and the second sealed trays are sealed with a polyethylene based film.

18. The kit of claim 17, wherein the polyethylene based film is compatible to sterilization with ethylene dioxide.

19. A kit to facilitate easy, efficient, and sterile refilling of baclofen in an implantable intrathecal pump, the kit comprising a sealed setup tray comprising items for removing any residual baclofen from the pump, and a sealed medication tray comprising items for refilling the pump with fresh baclofen, wherein
the sealed setup tray comprises layers in the following order:
a) a first setup tray layer facing the user and comprising a topical antiseptic solution wipes and applicators,
b) a second setup tray layer positioned below the first setup tray layer and comprising sterile disposable gloves,
c) a third setup tray layer positioned below the second setup tra layer and comprising a fenestrated drape, and
d) a fourth setup tray layer positioned below the third setup tray layer and comprising a syringe, a needle, clampable tubing, and a sharps plug; and
the sealed medication tray comprises layers in the following order:
a) a first medication tray layer facing the user and comprising a syringe, prescribing information, and information for use,
b) a second medication tray layer positioned below the first medication tray layer and comprising at least gauze pad or a gauze roll,
c) a third medication tray layer positioned below the second medication tray layer and comprising at least one polybag, and
d) a fourth medication tray layer beneath the third medication tray layer comprising at least one gauze pad or a roll and a label,
e) a fifth medication tray layer positioned below the fourth layer and comprising medication ampule, aspiration tube, ampule opener, and a syringe cap.
wherein the setup tray and the medication trays are sterilized after sealing to keep the outer surface of the ampule and the syringes sterile during use, and
wherein the order of layers in the setup tray and the medication tray is based on requirement of supplies during each step of refilling the pump.
